(19) United States
(12) Patent Application Publication
(10) Pub. No.: US 2025/0140381 A1
(43) Pub. Date: May 1, 2025

(54) MODALITY APPARATUS, INFORMATION PROCESSING METHOD, PROGRAM, AND MEDICAL INFORMATION SYSTEM

(71) Applicant: FUJIFILM Corporation, Tokyo (JP)

(72) Inventor: Kazuya MIYAMOTO, Tokyo (JP)

(73) Assignee: FUJIFILM Corporation, Tokyo (JP)

(21) Appl. No.: 18/925,056

(22) Filed: Oct. 24, 2024

(30) Foreign Application Priority Data
Oct. 31, 2023 (JP) 2023-186819

(51) Int. Cl. G16H 30/20; G16H 30/40

(52) U.S. Cl. CPC G16H 30/20; G16H 30/40

(57) ABSTRACT

Provided are a modality apparatus, an information processing method, a program, and a medical information system including the modality apparatus, which can generate an examination order based on information on the latest protocol. A modality apparatus that captures a medical image of a subject according to an examination order includes: a storage device that stores an imaging protocol; and one or more processors configured to, in a case where a combination of intra-imaging protocol sequences of the imaging protocol consisting of a combination of a plurality of sequences is changed, execute processing of providing intra-modality information including the changed information to an information system that generates the examination order in a format defined between the information system and the modality apparatus.

### CROSS-REFERENCE TO RELATED APPLICATION

The present application claims priority under 35 U.S.C § 119 (a) to Japanese Patent Application No. 2023-186819 filed on Oct. 31, 2023, which is hereby expressly incorporated by reference, in its entirety, into the present application.

### BACKGROUND OF THE INVENTION

### 1. Field of the Invention

The present disclosure relates to a modality apparatus, an information processing method, a program, and a medical information system.

### 2. Description of the Related Art

In a case where an image diagnostic examination is performed in a medical facility such as a hospital, for example, a user such as a doctor generates an examination order by a radiology information system (RIS), the RIS receives a request from a modality to send the examination order to the modality from the RIS, and a user such as an examination technician performs an imaging operation of a patient while viewing the examination order in the modality.

JP2020-187542A discloses an imaging support device comprising a determination unit that determines an imaging condition in a case where imaging of a patient is executed by a modality in a form of an imaging protocol that is readable by the modality, based on contents of patient information related to the patient, order information for requesting imaging of the patient, and instruction information related to the execution of the imaging created according to the order information, and an output unit that outputs the imaging protocol determined by the determination unit to the modality.

In the technique disclosed in JP2020-187542A, machine learning is performed using a set of change factors of patient information, an examination order, and examination instruction acquired in the past as learning data and using an imaging protocol employed in the learning data as training data, thereby creating a trained model that infers an imaging protocol from the set of change factors of patient information, an examination order, and an examination instruction. Then, during operation, the RIS inputs the set of change factors into the trained model, obtains the imaging protocol as an output, and transmits the imaging protocol to the modality. In addition, JP2020-187542A describes a configuration in which, in a case where the imaging protocol transmitted from the RIS and an actually used imaging protocol do not match, for example, in a case where the imaging protocol is changed on the modality side, a correspondence relationship between the examination order and the imaging protocol is updated.

### SUMMARY OF THE INVENTION

In the technique disclosed in JP2020-187542A, it is checked whether the imaging protocol transmitted from the RIS after the imaging and the imaging protocol actually used on the modality side match. Therefore, there is a problem in that the RIS cannot ascertain the change in the protocol on the modality side until imaging with the changed protocol is performed. In a case where the RIS does not ascertain the latest protocol, an appropriate examination order cannot be issued.

The present disclosure has been made in view of such circumstances, and an object of the present disclosure is to provide a modality apparatus, an information processing method, a program, and a medical information system including the modality apparatus, which can generate an examination order based on information on the latest protocol.

According to a first aspect of the present disclosure, there is provided a modality apparatus that captures a medical image of a subject according to an examination order, the modality apparatus comprising: a storage device that stores an imaging protocol; and one or more processors configured to, in a case where a combination of intra-imaging protocol sequences of the imaging protocol consisting of a combination of a plurality of sequences is changed, execute processing of providing intra-modality information including the changed information to an information system that generates the examination order in a format defined between the information system and the modality apparatus.

According to the first aspect, in a case where there is a change in the combination of the intra-imaging protocol sequences of the imaging protocol held in the modality apparatus, the changed intra-modality information including the changed information is provided from the modality apparatus to the information system according to a format defined between the modality apparatus and the information system. As a result, in the information system, it is possible to generate the examination order by using the provided latest intra-modality information.

The description that the information is provided to the information system includes a concept of putting the information system in a state in which the information can be acquired by the information system, regardless of whether the information is actually acquired by the information system.

According to a second aspect, in the modality apparatus according to the first aspect, the processing of providing the intra-modality information to the information system may include processing of storing the intra-modality information in a storage region accessible by the information system according to the format.

The storage region accessible by the information system may be provided in the modality apparatus, may be provided in the information system, or may be provided in another device.

According to a third aspect, in the modality apparatus according to the second aspect, the one or more processors may be configured to notify the information system in a case where new information is stored in the storage region.

According to a fourth aspect, in the modality apparatus according to any one of the first to third aspects, the one or more processors may be configured to provide information on a sequence that is addable to the imaging protocol to the information system.

According to a fifth aspect, in the modality apparatus according to the fourth aspect, the one or more processors may be configured to provide the addable sequence and an applicable examination part in association with each other to the information system.

According to a sixth aspect, in the modality apparatus according to any one of the first to fifth aspects, imaging protocol information defining the combination of the intra-imaging protocol sequences of the imaging protocol may be stored in the storage device, and the one or more processors may be configured to, in a case where the examination order that includes information on an intra-imaging protocol sequence different from the combination of the intra-imaging protocol sequences included in the imaging protocol information in the storage device is acquired, create an imaging protocol consisting of a combination of intra-imaging protocol sequences designated in the examination order as a new imaging protocol, and store the new imaging protocol in the storage device.

According to a seventh aspect, in the modality apparatus according to the sixth aspect, the one or more processors may be configured to: notify an operator of the modality apparatus in a case of storing the new imaging protocol; and store the new imaging protocol by assigning a new name different from an imaging protocol name of the imaging protocol already stored in the storage device.

According to an eighth aspect, in the modality apparatus according to any one of the first to seventh aspects, imaging protocol information defining a combination of the intra-imaging protocol sequences of the imaging protocol and sequence information including information on a sequence that is addable to the imaging protocol may be stored in the storage device, and the one or more processors may be configured to: provide the imaging protocol information and the sequence information to the information system according to the format; and in a case where at least one of the imaging protocol information or the sequence information is changed, provide the changed information to the information system according to the format.

According to a ninth aspect, there is provided a medical information system comprising: the modality apparatus according to any one of the first to eighth aspects; and an information system that issues the examination order.

According to a tenth aspect, there is provided an information processing method executed by a modality apparatus that captures a medical image of a subject according to an examination order, the information processing method comprising: storing an imaging protocol in a storage device by one or more processors of the modality apparatus; and in a case where a combination of intra-imaging protocol sequences of the imaging protocol consisting of a combination of a plurality of sequences is changed, executing processing of providing intra-modality information including the changed information to an information system in a format defined between the information system and the modality apparatus, by the one or more processors.

The information processing method according to the tenth aspect can be configured to include the same specific aspects as the modality apparatus according to the second to eighth aspects.

According to an eleventh aspect, there is provided a program for causing a computer used in a modality apparatus that captures a medical image of a subject according to an examination order to realize: a function of storing an imaging protocol in a storage device; and a function of, in a case where a combination of intra-imaging protocol sequences of the imaging protocol consisting of a combination of a plurality of sequences is changed, providing intra-modality information including the changed information to an information system that generates the examination order in a format defined between the information system and the modality apparatus.

The program according to the eleventh aspect can be configured to include the same specific aspects as the modality apparatus according to the second to eighth aspects.

The present disclosure also includes a non-transitory computer-readable recording medium (computer-readable medium) which is a tangible object on which the program according to the eleventh aspect is recorded, such as an optical disk, a semiconductor memory, and a magnetic disk.

According to the present disclosure, the modality apparatus can provide the latest information on the imaging protocol to the information system that issues the examination order. The information system can generate an examination order including an imaging protocol suitable for an examination purpose based on the latest information provided from the modality apparatus.

### BRIEF DESCRIPTION OF THE DRAWINGS

FIG. 1 is a block diagram schematically showing a configuration of an exemplary medical information system.

FIG. 2 is a perspective view schematically showing a configuration of a magnetic resonance imaging (MRI) apparatus which is an example of a modality apparatus.

FIG. 3 is a block diagram showing an example of a hardware configuration of a console unit.

FIG. 4 is an explanatory diagram showing an outline of an operation by an RIS and the modality apparatus in the present embodiment.

FIG. 5 is an explanatory diagram showing an example of an intra-modality information file.

FIG. 6 is a conceptual diagram showing an example of a method of providing the intra-modality information file from the modality apparatus.

FIG. 7 is a flowchart showing a procedure of intra-imaging protocol sequence comparison processing executed by a modality apparatus that has received an examination order.

FIG. 8 is a block diagram showing a functional configuration of a console unit in the present embodiment.

### DESCRIPTION OF THE PREFERRED EMBODIMENTS

Hereinafter, a preferred embodiment of the present invention will be described in detail with reference to the accompanying drawings. In the following description and the accompanying drawings, the same components are denoted by the same reference numerals, and duplicated descriptions will not be repeated.

### Configuration Example Medical Information System

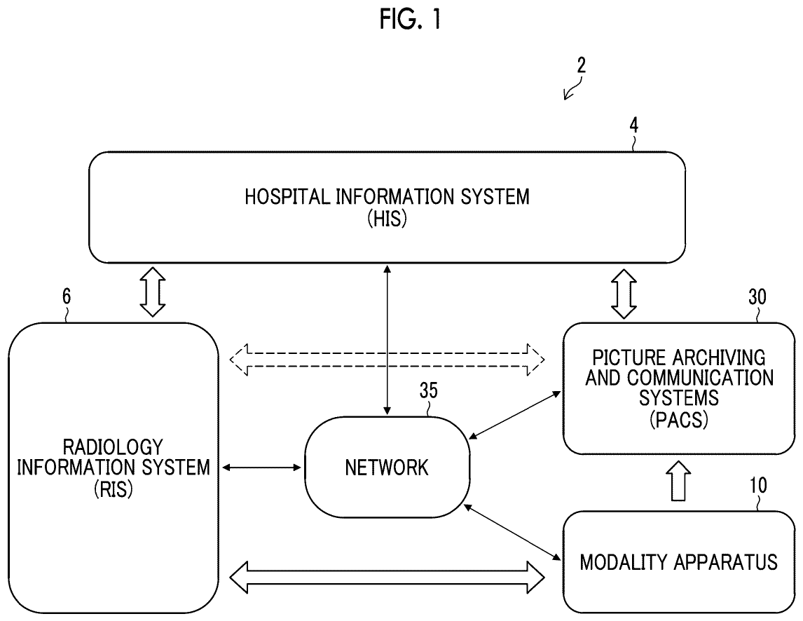

FIG. 1 is a block diagram schematically showing a configuration of an exemplary medical information system 2. The medical information system 2 is an information processing system introduced into a medical facility such as a hospital, and includes a hospital information system (HIS) 4, an RIS 6, a modality apparatus 10, and picture archiving and communication systems (PACS) 30.

The HIS 4 is a system that manages various types of tasks within the entire medical facility. The HIS 4 can be configured to include, for example, a plurality of systems such as an electronic medical record system, an ordering system, and a medical accounting system.

The RIS 6 is a system that manages tasks of a radiology department (radiology division). The RIS 6 cooperates with the HIS 4 to perform processing such as examination reservation of an image diagnostic examination, creation of an examination order, and management of examination results.

The modality apparatus 10 is an examination apparatus that captures a medical image of a subject undergoing an image diagnostic examination. Specific examples of the modality apparatus 10 include various examination apparatuses, such as a magnetic resonance imaging (MRI) apparatus, a computed tomography (CT) apparatus, an ultrasound diagnostic apparatus, a positron emission tomography (PET) apparatus, a mammography apparatus, an X-ray diagnostic apparatus, an X-ray fluoroscopic diagnostic apparatus, and an endoscope apparatus. In FIG. 1, one modality apparatus 10 is representatively shown, but the medical information system 2 may include a plurality of modality apparatuses 10. There can be various combinations of the type and the number of modality apparatus 10 for each medical facility.

The PACS 30 is a computer system that stores and manages various data including medical images obtained by the image diagnostic examination using the modality apparatus 10. The PACS 30 comprises a large-capacity external storage device and a database management program. The PACS 30 may be configured to include, for example, a digital imaging and communications in medicine (DICOM) server that operates in accordance with a DICOM specification.

The HIS 4, the RIS 6, the modality apparatus 10, and the PACS 30 are connected in a communicable manner via a network 35. The network 35 may be, for example, a local area network in a medical facility. A part of the network 35 may be a wide area network.

The PACS 30 receives various data including images generated by the modality apparatus 10 via the network 35, stores the data in a recording medium such as a large-capacity external storage device, and manages the data. The PACS 30 communicates with other devices, such as the HIS 4 and the RIS 6, via the network 35 to transmit and receive various data including medical images. In addition, a storage format of image data in the PACS 30 and the communication between the respective devices via the network 35 are based on, for example, a DICOM protocol.

### Example of Modality Apparatus 10: MRI Apparatus

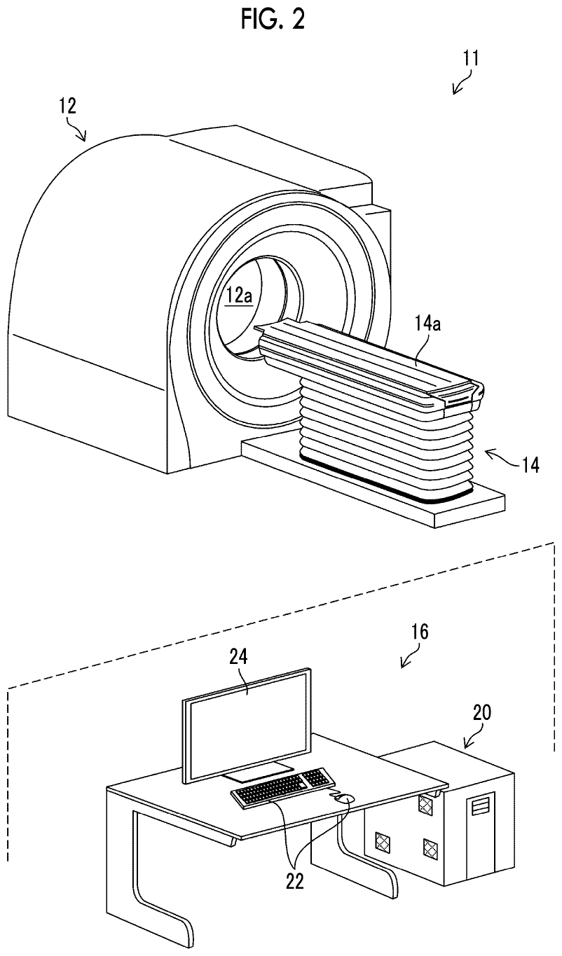

FIG. 2 is a perspective view schematically showing a configuration of an MRI apparatus 11, which is an example of the modality apparatus 10. The MRI apparatus 11 comprises a measurement unit 12 including a gantry, an examination table 14, and a console unit 16. The measurement unit 12 and the examination table 14 are disposed in an imaging room, and the console unit 16 is disposed in an operation room. In addition, although not shown, the MRI apparatus 11 comprises a gradient magnetic field power supply unit, a radio frequency (RF) amplifier unit, a helium compressor unit, a control unit, and the like. These units (not shown) are stored in a housing as individual units or as a combination of a plurality of units, and are disposed in a machine room. Some or all of these units may be disposed in the operation room.

Although a detailed configuration of the measurement unit 12 is not shown, the measurement unit 12 comprises a static magnetic field generation magnet, a gradient magnetic field coil, an RF irradiation coil, an RF reception coil, and the like. The static magnetic field generation magnet is, for example, a superconducting magnet, and generates a static magnetic field in an imaging space 12a of the gantry. The gradient magnetic field coil provides a magnetic field gradient to the static magnetic field generated by the static magnetic field generation magnet. The RF irradiation coil generates a high-frequency magnetic field in a measurement region of the subject. The RF reception coil receives a nuclear magnetic resonance (NMR) signal generated from the measurement region of the subject.

Since a detailed configuration of each part of the measurement unit 12 and an operation principle of the MRI apparatus 11 are known technologies, the specific description thereof will be omitted here.

The examination table 14 includes a top plate 14a on which a subject undergoing an image diagnostic examination is placed. The top plate 14a is movable in an up-down direction, a direction (forward direction) in which the top plate 14a enters the imaging space 12a of the gantry, and a direction (rearward direction) in which the top plate 14a exits the imaging space 12a, by a drive unit (not shown).

The console unit 16 includes an information processing apparatus 20, an input device 22, and a display device 24. The console unit 16 functions as a control device that controls a control unit of the machine room to control each part of the measurement unit 12 and the examination table 14 and executes MRI imaging to acquire an NMR signal. In addition, the console unit 16 functions as a device that performs processing of various types of data including processing of reconstructing images based on the NMR signal acquired from the measurement unit 12 and communication processing through the network 35, displaying processing results, storing data, and the like.

The NMR signal obtained from the measurement unit 12 is subjected to signal processing and then is subjected to digital image processing, and the reconstructed image is displayed on the display device 24 of the console unit 16.

### Hardware Configuration Example of Console Unit 16

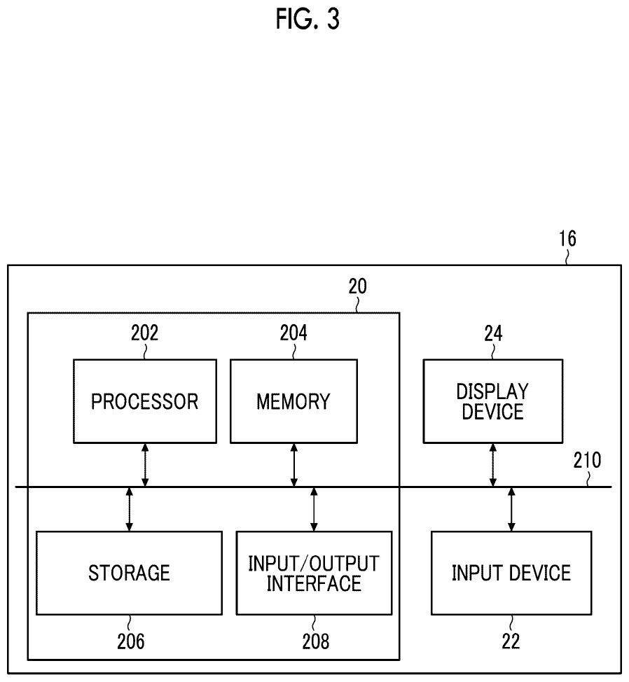

FIG. 3 is a block diagram showing an example of a hardware configuration of the console unit 16. The console unit 16 is configured using a computer. The computer applied to the console unit 16 may be a personal computer, a workstation, or a server computer. The processing function of the console unit 16 may be realized by a computer system including a plurality of computers.

The information processing apparatus 20 of the console unit 16 comprises a processor 202, a memory 204 which is a main storage device, a storage 206 which is an auxiliary storage device, an input/output interface 208, and a bus 210.

The processor 202 includes a central processing unit (CPU). The processor 202 may include a graphics processing unit (GPU). In addition, the information processing apparatus 20 may be configured to further include processors such as a digital signal processor (DSP), an application specific integrated circuit (ASIC), and a programmable logic device (PLD).

The processor 202 is connected to the memory 204, the storage 206, the input/output interface 208, the input device 22, and the display device 24 via the bus 210.

The memory 204 includes a random access memory (RAM). The memory 204 may include a read only memory (ROM). The storage 206 may be, for example, a hard disk drive (HDD), a solid state drive (SSD), or a combination of a plurality thereof. In addition, the storage 206 may include an external storage device such as a removable disk.

A storage device including the memory 204 and the storage 206 stores programs, data, and the like for realizing various functions of the MRI apparatus 11. The processor 202 realizes various functions by executing a program stored in the memory 204. The processor 202 integrally controls each part of the information processing apparatus 20 and various devices and units provided in the MRI apparatus 11, and performs various types of processing.

The input/output interface 208 includes a communication interface connectable to the network 35 and a connection interface connectable to an external device. As the connection interface connectable to the external device, for example, a universal serial bus, a high-definition multimedia interface (HDMI) (HDMI is a registered trademark), or the like can be applied.

The processor 202 communicates with various devices of the MRI apparatus 11 via the input/output interface 208, thereby transmitting and receiving necessary information.

The input device 22 is configured by, for example, a keyboard, a pointing device such as a mouse, a ten-key, and various switch buttons. The input device 22 may include a voice input device. In addition, the input device 22 may be a touch panel-type input device that is integrally configured with a display screen of the display device 24.

Various instructions or information input by an operator via the input device 22 are input to the information processing apparatus 20. The operator interactively operates the MRI apparatus 11 by using the input device 22 and the display device 24.

The display device 24 is configured of, for example, a liquid crystal display, an organic electro-luminescence (OEL) display, a projector, or an appropriate combination thereof. The display device 24 is connected to the information processing apparatus 20 via the bus 210. The display device 24 displays various types of information in addition to an examination image captured by the MRI apparatus 11. The display device 24 is used as a part of a user interface (UI) in a case of receiving input from the input device 22. In addition, the number of display devices 24 is not limited to one, and a multi-display configuration including a plurality of display devices is also possible.

### Outline of Mechanism for Sharing Latest Information on Imaging Protocol and Sequence Between Modality Apparatus 10 and RIS 6

In the present embodiment, in order to issue an accurate examination order in the RIS 6, means for allowing the RIS 6 to appropriately acquire the latest information on an imaging protocol and a sequence in the modality apparatus 10 is provided. In the present embodiment, an example is described in which the examination order is generated by the RIS 6, and the examination order is transmitted from the RIS 6 to the modality apparatus 10 after an examination order acquisition request from the modality apparatus 10 is received. However, a configuration may be adopted in which the examination order is generated by, for example, another information system such as an ordering system of the HIS 4, not limited to the RIS 6, and the examination order is transmitted to the modality apparatus 10. In this case, the information system that replaces the RIS 6 can acquire intra-modality information provided by the modality apparatus 10. The RIS 6 or the HIS 4 is an example of the information system.

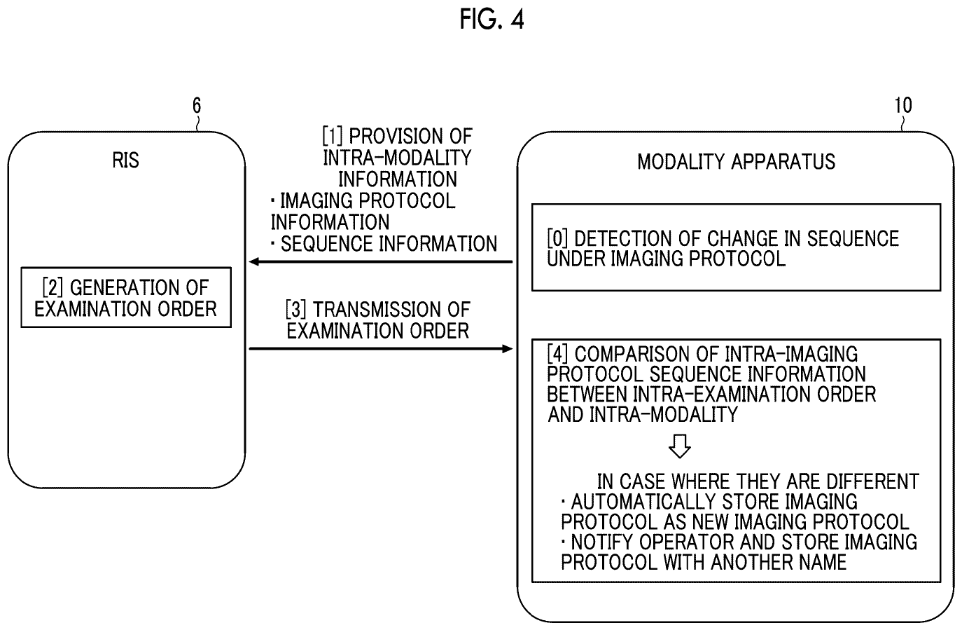

FIG. 4 is an explanatory diagram showing an outline of the operation by the RIS 6 and the modality apparatus 10 in the present embodiment. The present system basically operates in the following procedure ([Step 0]→[Step 1]→[Step 2]→[Step 3]→[Step 4]).

### [Step 0] Detection of Change in Sequence Under Imaging Protocol

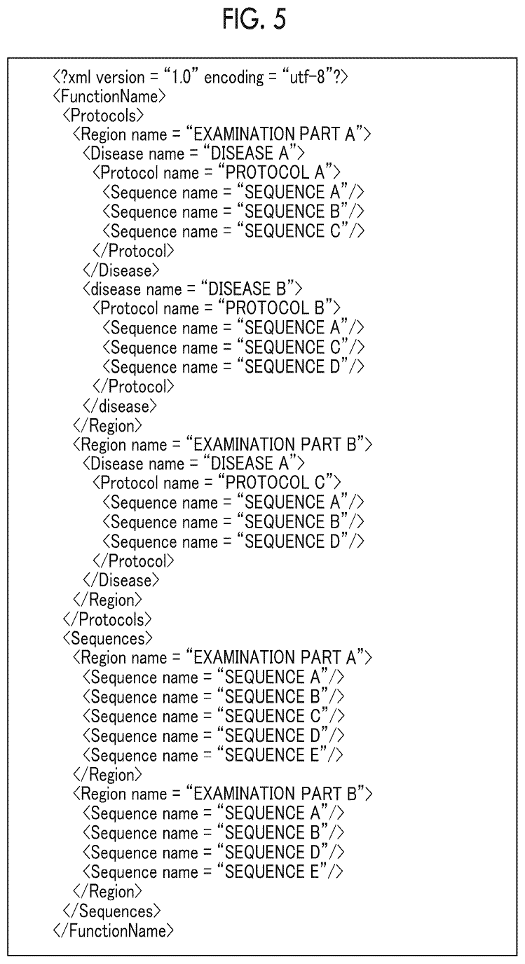

First, on the modality apparatus 10 side, it is detected that the sequence under the imaging protocol in the modality apparatus 10 is changed before examination order generation described in Step 3 which will be described later. Specifically, for example, in a scenario such as <1> new setting of a corresponding sequence in the imaging protocol due to new delivery of the modality apparatus 10 to a hospital or the like, <2> change in the corresponding sequence in the imaging protocol due to replacement of the user of the RIS 6, for example, a radiologist, <3> update of the modality apparatus 10, and <4> case of Step 4 described later, the modality apparatus 10 detects that an intra-modality information file of FIG. 5 described later has been rewritten in the modality apparatus 10, and compares and determines whether intra-imaging protocol sequence information is different before and after the rewriting.

The imaging protocol is composed of a combination of a plurality of sequences. The sequence included in the imaging protocol is referred to as an intra-imaging protocol sequence. In the modality apparatus 10, information on sequences that can be applied to each examination part is provided, and a plurality of imaging protocols consisting of combinations of various sequences can be defined in advance.

### [Step 1] Provision of Intra-Modality Information

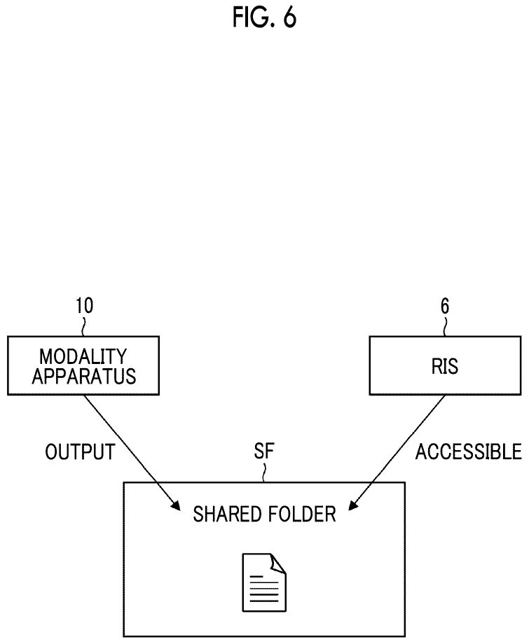

In addition to imaging protocol information and the intra-imaging protocol sequence information held in the storage device of the modality apparatus 10, the modality apparatus 10 provides the RIS 6 with information on addable sequences not included in the imaging protocol, in association with the applicable examination part (see FIGS. 5 and 6).

The information on the imaging protocol and the sequence stored in the storage device of the modality apparatus 10 is referred to as intra-modality information. The modality apparatus 10 provides the intra-modality information to the RIS 6 in a format defined between the modality apparatus 10 and the RIS 6. The intra-modality information provided from the modality apparatus 10 to the RIS 6 is the latest intra-modality information at that point in time.

### [Step 2] Generation of Examination Order

On the RIS 6 side, the examination order is generated using the information provided by the modality apparatus 10 in Step 1.

### [Step 3] Transmission of Examination Order

The examination order generated in the RIS 6 is transmitted from the RIS 6 to the modality apparatus 10 in response to an acquisition request from the modality apparatus 10.

### [Step 4] Comparison Processing of Intra-Imaging Protocol Sequence Information

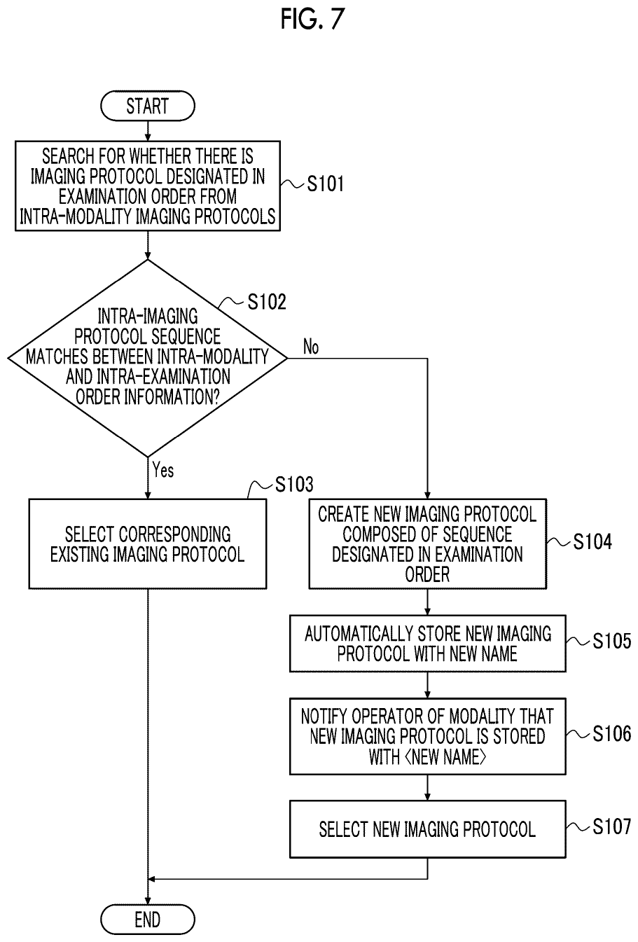

On the modality apparatus 10 side that has received the examination order, the intra-imaging protocol sequence information is compared between the intra-examination order and the intra-modality, and in a case where they are different, the imaging protocol designated in the examination order is automatically stored as a new imaging protocol. In this case, the modality apparatus 10 notifies the operator and stores the new imaging protocol under another name different from an imaging protocol name of the imaging protocol already stored in the modality apparatus 10 (see FIG. 7).

That is, the modality apparatus 10 compares the intra-imaging protocol sequence information held in the modality apparatus 10 with the intra-imaging protocol sequence information in the examination order acquired from the RIS 6, and in a case where they are different, the modality apparatus 10 automatically creates the imaging protocol designated in the examination order as a new imaging protocol. The operator of the modality apparatus 10 is notified of the newly created imaging protocol, and this protocol is automatically stored with an imaging protocol name different from that of the existing imaging protocol inside the modality apparatus 10.

The notification to the operator need only be a presentation of information indicating that the imaging protocol designated in the acquired examination order is different from the imaging protocol held in the modality apparatus 10 and is stored as a new imaging protocol, and a format of the notification is not particularly limited.

The intra-modality information including the information on the new imaging protocol stored by Step 4 is provided to the RIS 6 by Step 1.

[Step 2] to [Step 4] described above are executed each time for each examination. [Step 0] and [Step 1] are executed in a case where a change occurs in the intra-modality information. A method including Step 0, Step 1, and Step 4 shown in FIG. 4 is an example of an information processing method in the present disclosure. Hereinafter, further specific examples will be shown and described.

### Example of Method of Providing Intra-Modality Information

The modality apparatus 10 provides the intra-modality information to the RIS 6 in a format defined between the modality apparatus 10 and the RIS 6. The modality apparatus 10 provides the intra-modality information, for example, by a file in a predetermined file format.

FIG. 5 is an explanatory diagram showing an example of the intra-modality information file. FIG. 5 shows an example in which the imaging protocol information and the intra-imaging protocol sequence information in the modality apparatus 10 as well as sequence information that can be added or changed are provided by an extensible markup language (XML) file. This file includes, for each examination part, information on the imaging protocol name associated with single screening of each disease or healthy person and what kind of sequence (intra-imaging protocol sequence) is included under the imaging protocol, as well as a list of sequences that can be additionally set.

Specific names are described in each of name parts such as “examination part A,” “disease A,” and “sequence A” in FIG. 5. For the sequence name, for example, an abstract name such as “diffusion-weighted image” may be used to easily determine what kind of sequence it is on the RIS 6 side.

In addition, since a target examination part is decided in advance for the additional sequence, it is desirable to provide information from the modality apparatus 10 to the RIS 6 by associating the target examination part with the additional sequence such that a relationship between the target examination part and the sequence that can be added and changed is understood.

In the example shown in FIG. 5, the imaging protocol is described in association with single screening of each disease or healthy person corresponding to the examination part, but is not limited to this. The imaging protocol may be directly associated with the examination part or may be associated with a disease name regardless of the examination part.

In addition, in FIG. 5, the XML file is described as an example, but the information may be provided in another format that can be read on the RIS 6 side, not limited to the XML file. The XML format is an example of a format defined between the RIS 6 and the modality apparatus 10.

FIG. 6 is a conceptual diagram showing an example of a method of providing the intra-modality information file from the modality apparatus 10. The processor 202 of the modality apparatus 10 outputs the intra-modality information file to a location accessible by the RIS 6 such as a shared folder SF. The shared folder SF in which the intra-modality information file is stored may be provided in the storage device of the modality apparatus 10, may be provided in the storage device on the RIS 6 side, or may be provided in a storage device of another apparatus (system) other than these. The shared folder SF is an example of a storage region accessible by the RIS 6.

The processor 202 of the modality apparatus 10 may comprise a mechanism for issuing a notification to the RIS 6 in a case where the intra-modality information file is stored in the shared folder SF. The RIS 6 can access the shared folder SF to acquire the latest intra-modality imaging protocol information and sequence information.

In a case where a combination of the intra-imaging protocol sequences of the imaging protocol is changed, the processor 202 of the modality apparatus 10 stores the intra-modality information file including the changed information, in the shared folder SF. In addition, a case where a new imaging protocol is created corresponds to a case where the combination of the intra-imaging protocol sequences of the imaging protocol is changed. In addition, for example, in a case where there is a change in at least one of the imaging protocol information or the sequence information in the modality apparatus 10, such as a case where a part of a plurality of imaging protocols held in the modality apparatus 10 is deleted, a case where an imaging protocol name of an existing imaging protocol is changed, a case where an addable sequence is added, or a case where a part of a plurality of addable sequences held in the modality apparatus 10 is deleted, the processor 202 stores the intra-modality information file including the changed information in the shared folder SF.

The processor 202 may update the intra-modality information file in the shared folder SF to the latest file, or may store the latest file while leaving the previous file. It is sufficient that the RIS 6 can acquire the latest information from the shared folder SF, and the processor 202 may provide the changed information as a difference file according to a predetermined format.

The RIS 6 generates the examination order by using the information provided by the modality apparatus 10.

Flow of Comparison Processing of Intra-imaging protocol Sequence in Modality Apparatus 10

FIG. 7 is a flowchart showing a procedure of comparison processing of the intra-imaging protocol sequence executed by the modality apparatus 10 that has received the examination order.

In step S101, the processor 202 searches for whether there is an imaging protocol designated in the examination order from among the imaging protocols (intra-modality imaging protocols) held in the modality apparatus 10.

In step S102, it is determined whether the intra-imaging protocol sequence matches between the intra-modality and the intra-examination order information.

In a case where a determination result in step S102 is Yes, that is, in a case where the intra-imaging protocol sequence designated in the examination order matches the intra-imaging protocol sequence held in the modality apparatus 10 as a result of determination in step S102, the processor 202 proceeds to step S103.

In step S103, the processor 202 selects the existing imaging protocol corresponding to the designation of the examination order.

On the other hand, in a case where the determination result in step S102 is No, that is, in a case where the intra-imaging protocol sequence designated in the examination order does not match the intra-imaging protocol sequence held in the modality apparatus 10 as a result of determination in step S102, the processor 202 proceeds to step S104.

In step S104, the processor 202 creates a new imaging protocol composed of the sequence designated in the examination order.

In step S105, the processor 202 automatically stores the created new imaging protocol with a new name.

In step S106, the processor 202 notifies the operator of the modality apparatus 10 that the new imaging protocol is stored with an imaging protocol name of “<new name>”. In addition, before the storage of the new imaging protocol by step S105, the operator may be given a notification that the new imaging protocol is to be stored by indicating <new name>, and the new imaging protocol may be stored after the notification.

In step S107, the processor 202 automatically selects the new imaging protocol as the imaging protocol to be used for an examination instruction of the examination order.

After step S103 or step S107, the flowchart of FIG. 7 ends. The flowchart of FIG. 7 may be executed for each received examination order.

### Functional Configuration of Console Unit 16

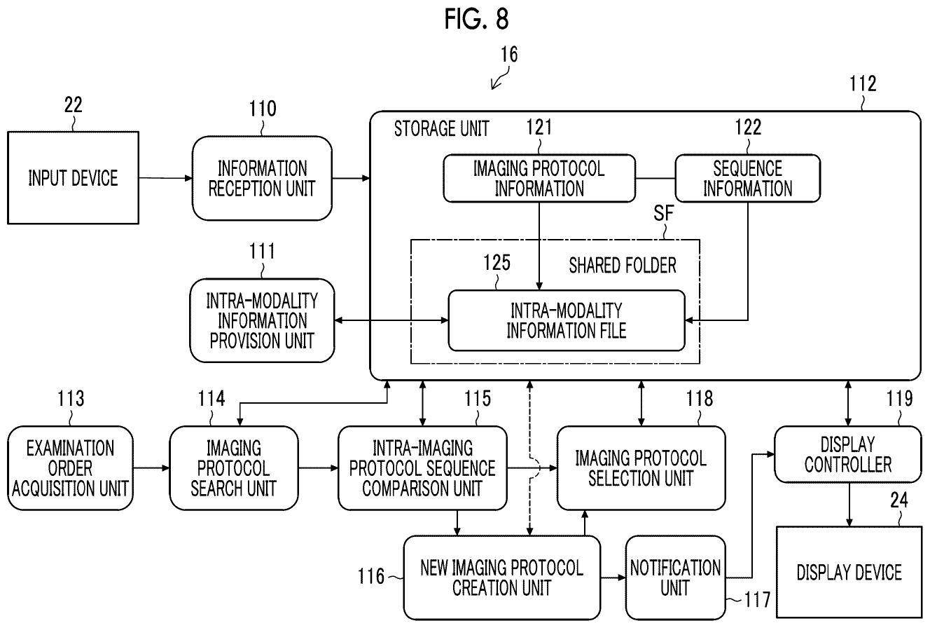

FIG. 8 is a block diagram showing a functional configuration of the console unit 16 in the present embodiment. The console unit 16 comprises an information reception unit 110, an intra-modality information provision unit 111, a storage unit 112, an examination order acquisition unit 113, an imaging protocol search unit 114, an intra-imaging protocol sequence comparison unit 115, a new imaging protocol creation unit 116, a notification unit 117, an imaging protocol selection unit 118, and a display controller 119.

Each of the information reception unit 110, the intra-modality information provision unit 111, the examination order acquisition unit 113, the imaging protocol search unit 114, the intra-imaging protocol sequence comparison unit 115, the new imaging protocol creation unit 116, the notification unit 117, the imaging protocol selection unit 118, and the display controller 119 may be a configuration that can be realized by executing a command stored in the memory 204 by the processor 202 shown in FIG. 3. The storage unit 112 may be a storage device including the memory 204 and the storage 206 shown in FIG. 3.

The information reception unit 110 receives input of information via the input device 22 and performs processing according to the received information. The information reception unit 110 performs, for example, processing of receiving an instruction to change the imaging protocol information or to change the sequence information and storing the changed information in the storage unit 112.

The intra-modality information provision unit 111 performs processing of providing the information on the imaging protocol and the sequence stored in the storage unit 112 to the RIS 6.

Imaging protocol information 121 and sequence information 122 are stored in the storage unit 112. The imaging protocol information 121 includes a plurality of imaging protocols for each examination part. One or more sequences are defined for each imaging protocol. The sequence information 122 includes information on additional sequences that can be added or changed for each examination part.

In addition, the storage unit 112 stores an intra-modality information file 125 generated by the intra-modality information provision unit 111.

The intra-modality information provision unit 111 generates the intra-modality information file 125 based on the imaging protocol information 121 and the sequence information 122. As shown in FIG. 5, the intra-modality information file 125 may be, for example, an XML file.

In the example shown in FIG. 8, the shared folder SF is provided in the storage unit 112, and the intra-modality information file 125 is stored in the shared folder SF.

In a case where at least a part of the imaging protocol information 121 and the sequence information 122 is updated, the intra-modality information provision unit 111 updates the intra-modality information file 125 based on the latest information. As a result, the latest intra-modality information is provided from the modality apparatus 10 to the RIS 6.

The examination order acquisition unit 113 acquires the examination order from the RIS 6. The imaging protocol search unit 114 searches for the imaging protocol designated in the acquired examination order from the imaging protocol information 121. The intra-imaging protocol sequence comparison unit 115 receives a search result from the imaging protocol search unit 114 and performs comparison processing between the intra-imaging protocol sequence in the modality apparatus 10 that is extracted by the search and a sequence obtained by combining the intra-imaging protocol sequence information designated in the examination order and the changed sequence information to determine whether these sequences match.

The new imaging protocol creation unit 116 performs processing of creating a new imaging protocol composed of the sequence designated in the examination order in a case where a comparison result of the intra-imaging protocol sequence comparison unit 115 is determined to be a mismatch and storing the new imaging protocol in the storage unit 112 by adding a new imaging protocol name.

The notification unit 117 performs processing of notifying the operator that the new imaging protocol has been stored. The information to be notified to the operator is displayed on display device 24 via display controller 119. In addition, the notification unit 117 performs processing of notifying the RIS 6 in a case where the new information is stored in the shared folder SF.

The imaging protocol selection unit 118 performs processing of selecting an appropriate imaging protocol corresponding to the examination instruction according to the information on the examination order. The imaging protocol selection unit 118 selects the existing imaging protocol held in the storage unit 112 in a case where a match is determined from the comparison result of the intra-imaging protocol sequence comparison unit 115. In a case where the comparison result of the intra-imaging protocol sequence comparison unit 115 is determined to be a mismatch, the imaging protocol selection unit 118 selects the imaging protocol newly created by the new imaging protocol creation unit 116.

The display controller 119 generates a display signal required for display output to the display device 24 and performs display control of the display device 24.

### Hardware Configuration of Each Processing Unit

A hardware structure of a processing unit that executes various types of processing, such as the information reception unit 110, the intra-modality information provision unit 111, the examination order acquisition unit 113, the imaging protocol search unit 114, the intra-imaging protocol sequence comparison unit 115, the new imaging protocol creation unit 116, the notification unit 117, the imaging protocol selection unit 118, and the display controller 119 shown in FIG. 8, is, for example, various processors shown below.

The various processors include a CPU that is a general-purpose processor functioning as various processing units by executing a program, a GPU, a programmable logic device (PLD) such as a field programmable gate array (FPGA) that is a processor having a circuit configuration changeable after manufacture, a dedicated electric circuit such as an application specific integrated circuit (ASIC) that is a processor having a circuit configuration dedicatedly designed to execute specific processing, and the like.

One processing unit may be configured by one of these various processors or may be configured by two or more processors of the same type or different types. For example, one processing unit may be composed of a plurality of FPGAs, a combination of a CPU and an FPGA, or a combination of a CPU and a GPU. A plurality of processing units may be configured by one processor. As an example in which the plurality of processing units are configured by one processor, first, there is a form in which one processor is configured by a combination of one or more CPUs and software, and this processor functions as the plurality of processing units, as represented by a computer, such as a client or a server. Second, as represented by a system-on-chip (SoC) or the like, there is a form in which a processor, which realizes the functions of the entire system including a plurality of processing units with one integrated circuit (IC) chip, is used. As described above, the various processing units are configured using one or more of the above various processors as a hardware structure.

Further, as the hardware structure of the various processors, more specifically, an electric circuit (circuitry) in which circuit elements such as semiconductor elements are combined is used.

### Effect of Embodiment

With the medical information system 2 including the modality apparatus 10 and the RIS 6 according to the present embodiment, the following effects can be obtained.

[1] The modality apparatus 10 can provide the latest information on the imaging protocol in the modality apparatus 10 to the RIS 6 that issues the examination order. The RIS 6 can issue an examination order including an appropriate examination instruction by using the latest information provided from the modality apparatus 10.

[2] In the modality apparatus 10, since setting contents related to the imaging protocol can be changed at any time, it is possible to flexibly respond to a change in the imaging protocol or a change in clinical situations after the system starts to operate. Since the information including the changed setting content is provided from the modality apparatus 10 to the RIS 6 each time the change is made, the RIS 6 can generate an appropriate examination order by using the latest setting information.

[3] In addition, in a case where the modality apparatus 10 has acquired an examination order that includes an imaging protocol consisting of a combination of sequences different from the imaging protocol held in the modality apparatus 10, the modality apparatus 10 can automatically store the imaging protocol in the examination order as a new imaging protocol and provide information on the new imaging protocol to the RIS 6. As a result, thereafter, it becomes easier to create an examination order that includes the designation of the new imaging protocol in the RIS 6, thereby reducing a burden on a user such as a doctor.

### Modification Example

In the above-described embodiment, an example has been described in which the processor 202 of the modality apparatus 10 executes the processing of storing the intra-modality information file 125 in the shared folder SF that is accessible by the RIS 6. However, processing of providing the intra-modality information is not limited to this example. For example, the processor 202 may execute processing of writing the intra-modality information into a medium to provide the intra-modality information to the RIS 6, or processing of transmitting the intra-modality information to the RIS 6 via the network 35.

Regarding Program that Operates Computer

A program for causing a computer to realize some or all of processing functions of the information processing apparatus 20 in the modality apparatus 10 according to the present embodiment can be recorded on a computer-readable medium that is a non-transitory information storage medium such as an optical disk, a magnetic disk, a semiconductor memory, or other tangible object, and provide the program through this information storage medium.

Also, instead of an aspect in which the program is stored in such a non-transitory computer-readable medium which is a tangible object and provided, a program signal can be provided as a download service by using an electric telecommunication line such as the Internet.

Further, some or all of the processing functions in the information processing apparatus 20 may be realized by cloud computing or may be provided as software as a service (SaaS).

### Others

The present disclosure is not limited to the above-described embodiment, and various modifications can be made without departing from the spirit of the technical idea of the present disclosure.

### EXPLANATION OF REFERENCES

2: medical information system
4: hospital information system (HIS)
6: radiology information system (RIS)
10: modality apparatus
11: MRI apparatus
12: measurement unit
12a: imaging space
14: examination table
14a: top plate
16: console unit
20: information processing apparatus
22: input device
24: display device
30: picture archiving and communication systems (PACS)
35: network
110: information reception unit
111: intra-modality information provision unit
112: storage unit
113: examination order acquisition unit
114: imaging protocol search unit
115: intra-imaging protocol sequence comparison unit
116: new imaging protocol creation unit
117: notification unit
118: imaging protocol selection unit
119: display controller
121: Imaging protocol information
122: sequence information
125: intra-modality information file
202: processor
204: memory
206: storage
208: input/output interface
210: bus
S101 to S107: steps of comparison processing of intra-imaging protocol sequence in modality apparatus

What is claimed is:

1. A modality apparatus that captures a medical image of a subject according to an examination order, the modality apparatus comprising:
a storage device that stores an imaging protocol; and
one or more processors configured to, in a case where a combination of intra-imaging protocol sequences of the imaging protocol consisting of a combination of a plurality of sequences is changed, execute processing of providing intra-modality information including the changed information to an information system that generates the examination order in a format defined between the information system and the modality apparatus.

2. The modality apparatus according to claim 1,
wherein the processing of providing the intra-modality information to the information system includes processing of storing the intra-modality information in a storage region accessible by the information system according to the format.

3. The modality apparatus according to claim 2,
wherein the one or more processors are configured to notify the information system in a case where new information is stored in the storage region.

4. The modality apparatus according to claim 1,
wherein the one or more processors are configured to provide information on a sequence that is addable to the imaging protocol to the information system.

5. The modality apparatus according to claim 4,
wherein the one or more processors are configured to provide the addable sequence and an applicable examination part in association with each other to the information system.

6. The modality apparatus according to claim 1,
wherein imaging protocol information defining the combination of the intra-imaging protocol sequences of the imaging protocol is stored in the storage device, and
the one or more processors are configured to, in a case where the examination order that includes information on an intra-imaging protocol sequence different from the combination of the intra-imaging protocol sequences included in the imaging protocol information in the storage device is acquired, create an imaging protocol consisting of a combination of intra-imaging protocol sequences designated in the examination order as a new imaging protocol, and store the new imaging protocol in the storage device.

7. The modality apparatus according to claim 6,
wherein the one or more processors are configured to:
notify an operator of the modality apparatus in a case of storing the new imaging protocol; and
store the new imaging protocol by assigning a new name different from an imaging protocol name of the imaging protocol already stored in the storage device.

8. The modality apparatus according to claim 1,
wherein imaging protocol information defining a combination of the intra-imaging protocol sequences of the imaging protocol and sequence information including information on a sequence that is addable to the imaging protocol are stored in the storage device, and
the one or more processors are configured to:
provide the imaging protocol information and the sequence information to the information system according to the format; and
in a case where at least one of the imaging protocol information or the sequence information is changed, provide the changed information to the information system according to the format.

9. A medical information system comprising:
the modality apparatus according to claim 1; and
an information system that issues the examination order.

10. An information processing method executed by a modality apparatus that captures a medical image of a subject according to an examination order, the information processing method comprising:
storing an imaging protocol in a storage device by one or more processors of the modality apparatus; and
in a case where a combination of intra-imaging protocol sequences of the imaging protocol consisting of a combination of a plurality of sequences is changed, executing processing of providing intra-modality information including the changed information to an information system that generates the examination order in a format defined between the information system and the modality apparatus, by the one or more processors.

11. A non-transitory, computer-readable tangible recording medium which records thereon a program for causing, when read by a computer, the computer used in a modality apparatus that captures a medical image of a subject according to an examination order to realize:
a function of storing an imaging protocol in a storage device; and
a function of, in a case where a combination of intra-imaging protocol sequences of the imaging protocol consisting of a combination of a plurality of sequences is changed, providing intra-modality information including the changed information to an information system that generates the examination order in a format defined between the information system and the modality apparatus.
